(19) United States
(12) Patent Application Publication
(10) Pub. No.: US 2026/0345725 A1
(43) Pub. Date: Nov. 5, 2026

(54) METHOD OF TREATING OR PREVENTING DIABETES USING ENDOTHELIN-B RECEPTOR AGONIST OR SALT THEREOF

(71) Applicant: PHARMAZZ, INC., Willowbrook, IL (US)

(72) Inventor: Anil GULATI, Naperville, IL (US)

(21) Appl. No.: 19/198,401

(22) Filed: May 5, 2025

(51) Int. Cl. A61K 38/10; A61K 45/06; A61K 47/02; A61K 47/12; A61K 47/26; A61K 47/69; A61P 3/10

(52) U.S. Cl. CPC A61K 38/10; A61K 45/06; A61K 47/02; A61K 47/12; A61K 47/26; A61K 47/6929; A61P 3/10

(57) ABSTRACT

The disclosure relates to a method of treating or preventing diabetes and its complications comprising administering an endothelin-B receptor agonist or its pharmaceutically acceptable salt thereof. More specifically, the present disclosure relates to a method of treating or preventing hyperglycemia comprising administering an endothelin-B receptor agonist or its pharmaceutically acceptable salt thereof. The disclosure further relates to a pharmaceutical composition comprising an endothelin-B receptor agonist or its pharmaceutically acceptable salt thereof for treating or preventing diabetes in a subject in need thereof.

### FIELD

The present disclosure relates to a method of treating or preventing diabetes. More specifically, the present disclosure relates to methods of treating or preventing various types of diabetes comprising administering an endothelin-B receptor agonist or its pharmaceutically acceptable salt thereof. The disclosure further relates to a pharmaceutical composition comprising an endothelin-B receptor agonist or its pharmaceutically acceptable salt thereof for treating or preventing diabetes in a subject in need thereof.

### BACKGROUND

This disclosure relates to the use of an endothelin-B receptor agonist or its pharmaceutically acceptable salt thereof for the treatment of diabetes and related disorders as described herein.

In recent times, endothelin-B receptor agonists have gained large interest from the research community due to their good therapeutic effect in various neurological and cardiovascular diseases. The binding of an agonist to an endothelin-B receptors may result in activation of receptors, which further facilitate the regulation of vascular and neural functions. The use of endothelin-B receptor agonists in many neurological and cerebrovascular diseases is known from many literature, such as A. Raiet al. (2003) Cancer Chemother Pharmacol 51(1):21-8; A. Gulati (2003) U.S. Patent Publication 2004/0138121; and A. Gulati (2006) U.S. Patent Publication 2006/0211617); Administration of BQ788, a highly selective ET, receptor antagonist, blocked the tumor perfusion induced by IRL 1620 and confirmed the involvement of ET, receptors in tumor vasodilation (A. Rai et al. (2005); JPharm Pharmacol 57(7):869-76; N. V. Rajeshkumar et al. (2005) Breast Cancer Research and Treatment 94(3):237-247), the entire content of which is incorporated herein by reference.

The present disclosure is directed to a new use for endothelin-B receptor agonists in treating or preventing diabetes and related disorders. Surprisingly, it has now been found that an endothelin-B receptor agonist helps to lower blood glucose in diabetic patients and can, therefore, be useful in treating or preventing diabetes and related disorders.

### SUMMARY

The present disclosure provides methods of treating or preventing diabetes comprising administering an endothelin-B receptor agonist or its pharmaceutically acceptable salt thereof.

In one aspect, the present disclosure provides a method of treating or preventing diabetes in a subject in need thereof comprising administering an endothelin-B receptor agonist or its pharmaceutically acceptable salt thereof.

In one aspect, diabetes is all types of diabetes and its complications.

In another aspect, the present disclosure provides a method of treating or preventing diabetes or a disease or disorder caused by diabetes and its complications in a subject in need thereof comprising administering an endothelin-B receptor agonist or its pharmaceutically acceptable salt thereof.

In yet another aspect, the present disclosure provides a method of treating or preventing hyperglycemia in a subject in need thereof comprising administering an endothelin-B receptor agonist or its pharmaceutically acceptable salt thereof.

In one aspect, the present disclosure provides a method of treating or preventing diabetes and its complications in a subject in need thereof comprising administering sovateltide or salt thereof.

In another aspect, the present disclosure provides a method of treating or preventing diabetes and its complications or a disease or disorder caused by diabetes in a subject in need thereof comprising administering sovateltide or salt thereof.

In yet another aspect, the present disclosure provides a method of treating or preventing diabetes and its complications in a subject in need, comprising administering sovateltide or salt thereof.

In yet another aspect, the present disclosure provides a method of treating or preventing diabetes or a disease or disorder caused by diabetes in a subject in need thereof, comprising administering an endothelin-B receptor agonist or its pharmaceutically acceptable salt thereof, wherein the endothelin-B receptor agonist or its pharmaceutically acceptable salt thereof is administered in an amount sufficient to reduce or ameliorate one or more symptoms of said diabetes or disease or disorder caused by diabetes and its complications, in said individual, as compared to the individual prior to said administration.

In one aspect, the present disclosure provides a pharmaceutical composition comprising an endothelin-B receptor agonist or its pharmaceutically acceptable salt thereof for treating or preventing diabetes in a subject in need thereof.

In another aspect, the present disclosure provides a pharmaceutical composition comprising an endothelin-B receptor agonist or its pharmaceutically acceptable salt thereof for treating or preventing diabetes or a disease or disorder caused by diabetes and its complications in a subject in need thereof.

In yet another aspect, the present disclosure provides a pharmaceutical composition comprising an endothelin-B receptor agonist or its pharmaceutically acceptable salt thereof for treating or preventing diabetes and its complications in a subject in need thereof.

In one aspect, the present disclosure provides a pharmaceutical composition comprising sovateltide or salt thereof for treating or preventing diabetes and its complications in a subject in need thereof.

In another aspect, the present disclosure provides a pharmaceutical composition comprising sovateltide or salt thereof for treating or preventing diabetes or a disease or disorder caused by diabetes and its complications in a subject in need thereof.

In yet another aspect, the present disclosure provides a pharmaceutical composition comprising sovateltide or salt thereof for treating or preventing diabetes and its complications in a subject in need thereof.

In another aspect, the disclosure relates to a pharmaceutical composition intended for the treatment or prevention of diabetes and diabetes-related disorders. The composition comprises (a) an ETB receptor agonist or its analogues in a specified amount (b) at least one anti-diabetic medicament and (c) one or more pharmaceutically acceptable excipients.

The details of one or more embodiments of the present disclosure are outlined in the description below. Other features, objects, and advantages of the disclosure will be apparent from the description.

The aspects of the disclosed embodiments relate to a pharmaceutical formulation for the treatment of diabetes that is capable of being administered through a parenteral route, which can be an intravenous or intra-arterial route and works on a synergistic effect of pharmaceutically active ingredients in treating various types of diabetes.

According to an embodiment, a pharmaceutical formulation for diabetes treatment comprises an endothelin-B receptor analog in a dosing range of 0.00001 mg/kg-1 mg/kg.

According to another embodiment, the endothelin-B receptor analog includes, but not limited to, sovateltide, BQ-3020, [Ala^{1,3,11,15}]-endothelin, sarafotoxin S6c, endothelin-3.

While the aspects of the disclosed embodiments has been described and shown with particular reference to the preferred embodiment, it will be apparent that variations might be possible that would fall within the scope of the aspects of the disclosed embodiments.

### BRIEF DESCRIPTION OF THE DRAWINGS

These and other features, aspects, and advantages of the disclosed embodiments will become better understood with regard to the following description, appended claims, and accompanying drawings where:

FIG. 1 illustrates a graphical representation of blood glucose of all patients with or without diabetes at randomization (day 1) in the control and sovateltide groups.

FIG. 2 illustrates a graphical representation of blood glucose of patients who do not have diabetes and are treated with saline plus standard of care (control group).

FIG. 3 illustrates a graphical representation of blood glucose of patients who do not have diabetes and are treated with sovateltide plus standard of care (sovateltide group).

FIG. 4 illustrates a graphical representation of the blood glucose of all patients (diabetic plus non-diabetic) treated with saline plus standard of care (control group).

FIG. 5 illustrates a graphical representation of the blood glucose of all patients (diabetic plus non-diabetic) treated with sovateltide plus standard of care (sovateltide group).

FIG. 6 illustrates a graphical representation of the blood glucose of diabetic patients treated with saline plus standard of care (control group).

FIG. 7 illustrates a graphical representation of the blood glucose of diabetic patients treated with sovateltide plus standard of care (sovateltide group).

FIG. 8 illustrates a direct comparison of blood glucose of all patients (diabetic plus non-diabetic) treated with saline plus standard of care (control group) or sovateltide plus standard of care (sovateltide group).

FIG. 9 illustrates the change in blood cholesterol of non-diabetic and diabetic patients from day 1 to day 90 of treatment with sovateltide. Blood cholesterol is significantly lower at day 90 compared to day 1 following treatment with sovateltide.

### DESCRIPTION OF THE DRAWINGS

FIG. 1 illustrates a graphical representation of blood glucose of all patients with or without diabetes at randomization (day 1) in the control and sovateltide groups. Blood glucose is similar between the control and sovateltide groups (p=0.6599)

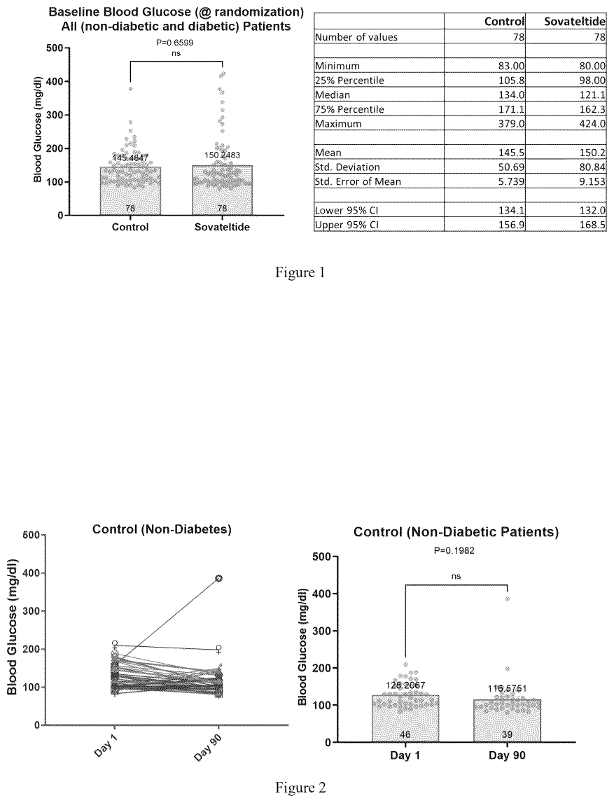

FIG. 2 illustrates a graphical representation of blood glucose of patients who do not have diabetes and are treated with saline plus standard of care (control group). Blood glucose has not changed from day 1 (baseline) to day 90 of treatment (p=0.1982);

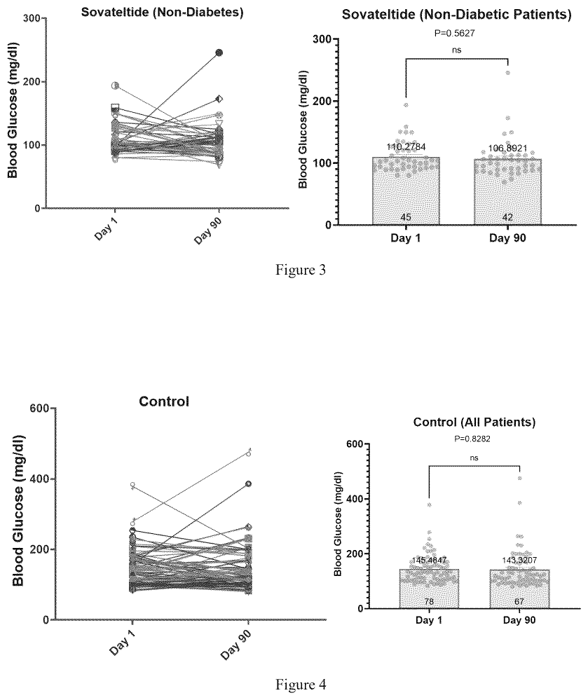

FIG. 3 illustrates a graphical representation of blood glucose of patients who do not have diabetes and are treated with sovateltide plus standard of care (sovateltide group). Blood glucose has not changed from day 1 (baseline) to day 90 of treatment (p=0.5627);

FIG. 4 illustrates a graphical representation of the blood glucose of all patients (diabetic plus non-diabetic) treated with saline plus standard of care (control group). Blood glucose has not changed from day 1 (baseline) to day 90 of treatment (p=0.8282);

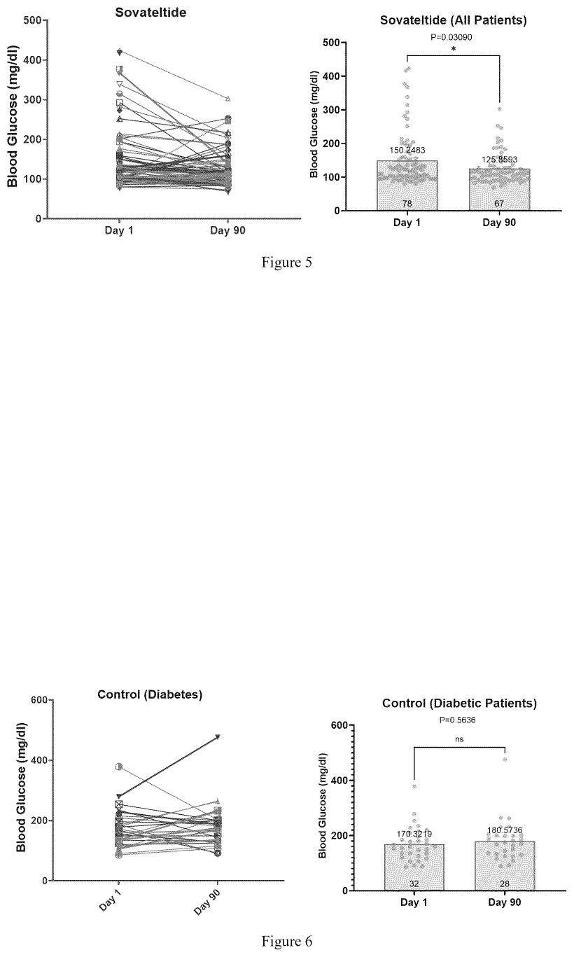

FIG. 5 illustrates a graphical representation of the blood glucose of all patients (diabetic plus non-diabetic) treated with sovateltide plus standard of care (sovateltide group). Blood glucose has significantly decreased from day 1 (baseline) to day 90 of treatment (p=0.03090);

FIG. 6 illustrates a graphical representation of the blood glucose of diabetic patients treated with saline plus standard of care (control group). Blood glucose has not changed from day 1 (baseline) to day 90 of treatment (p=0.5636);

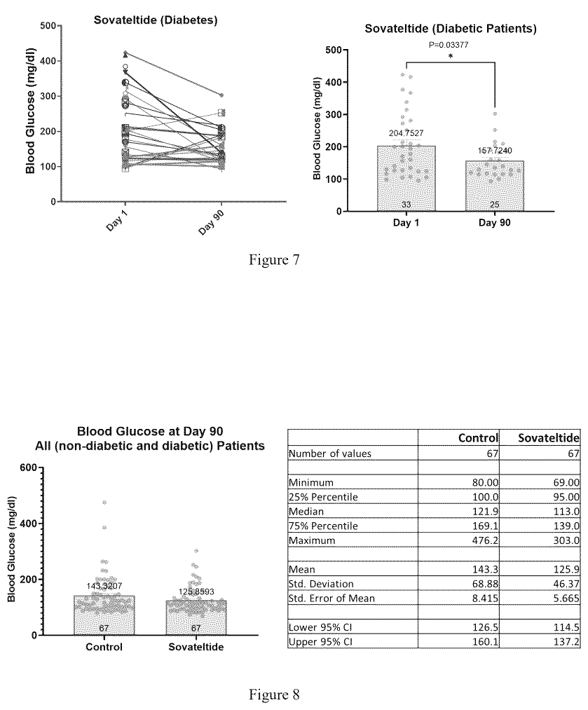

FIG. 7 illustrates a graphical representation of the blood glucose of diabetic patients treated with sovateltide plus standard of care (sovateltide group). Blood glucose has significantly decreased from day 1 (baseline) to day 90 of treatment (p=0.03377);

FIG. 8 illustrates a direct comparison of blood glucose of all patients (diabetic plus non-diabetic) treated with saline plus standard of care (control group) or sovateltide plus standard of care (sovateltide group). Blood glucose is lower in the sovateltide group than the control group at day 90 of treatment (−17.46, 95% CI −37.53 to 2.604);

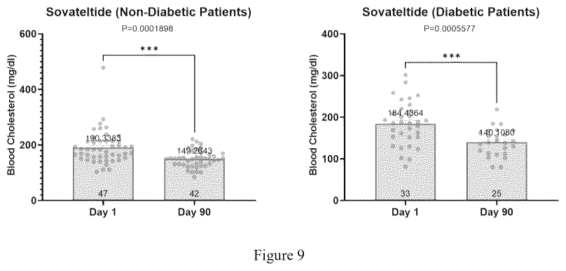

FIG. 9 illustrates the change in blood cholesterol of non-diabetic and diabetic patients from day 1 to day 90 of treatment with sovateltide. Blood cholesterol is significantly lower (non-diabetic p=0.0002; diabetic p=0.0005) at day 90 compared to day 1 following treatment with sovateltide.

### DETAILED DESCRIPTION

The disclosure may be more fully appreciated, and objects other than those set forth above will become apparent when consideration is given to the following description, including the glossary of terms and the concluding examples. The skilled artisan will appreciate that additional preferred embodiments may be selected by combining the preferred embodiments above or by reference to the examples given herein.

### Definitions

For the purpose of the present disclosure, the relevant terms are defined herein below:

As used herein, the terms “endothelin receptor agonist', “ET receptor agonist', “endothelin-B receptor agonist”, and “ET agonist” are used interchangeably. The endothelin-B receptor agonists disclosed herein are selected from a group comprising sovateltide, BQ-3020, [Ala1,3,11,15]-endothelin, sarafotoxin S6c, sarafotoxin 6c, or endothelin-3 or any combination thereof.

Sovateltide is one such endothelin receptor agonist having therapeutic potential for the treatment of such diseases/disorders, preferably neurological disorders. Sovateltide is a compound with 15 amino acid long chain. Sovateltide mimics the properties of a naturally occurring peptide called endothelin-1 (ET-1). ET-1 is known to activate two types of receptors in the body, namely endothelin A (ETARs) and endothelin B (ETBRs). Sovateltide is designed to activate the ETBRs specifically, making it a selective agonist for this receptor type. Various prior published patent documents disclose sovateltide, such as WO2008075379, WO2009026282, WO2015006324, WO2024/057296, and US 2024/0207356, the entire content of which is incorporated herein by reference.

The term ‘therapeutically effective amount’ indicates an amount of the compound that is effective in achieving the desired therapeutic result upon single or multiple dose administration(s) to a subject, in treating, curing, alleviating, relieving, or partially addressing the clinical manifestation of given disease or state and its complications beyond that expected in the absence of such treatment. Effective amounts of the active drug or compositions are dependent on any number of variables, including, without limitation, the species, gender, size, height, weight, age, the overall health of the patient, the type of formulation, or the mode or manner of administration. The appropriate, effective amount can be routinely determined by those skilled in the art using routine optimization techniques, the skilled and informed judgment of the practitioner, and other factors evident to those skilled in the art.

The phrase “pharmaceutically acceptable” is employed herein to refer to those compounds, materials, compositions, and/or dosage forms that are, within the scope of sound medical judgment.

As used in the specifications, the singular forms ‘a’, ‘an’, and ‘the’ include plural referents unless the context clearly dictates otherwise.

As used herein, the terms “comprising” and “comprises” mean the elements recited, or their equivalents in structure or function, plus any other element or elements which are not recited and the terms comprising the steps of include the steps recited, or the equivalent steps irrespective of the sequence in which they are recited.

The term “or” is used herein to mean, and is used interchangeably with, the term “and/or”, unless context clearly indicates otherwise.

The percentage(s) expressed herein are based on the final weight of the composition. As used herein, the term “about” means ±approximately 10% of the indicated value.

The term “subject” as used herein, refers to a warm-blooded animal such as a mammal, guinea pigs, dogs, cats, rats, mice, horses, cattle, sheep, rabbits, and humans are examples of mammals within the scope of the meaning of the term, preferably a human or a human child, which is afflicted with, or has the potential to be affected with one or more diseases and conditions described herein.

As used herein, “administering” or “administration” means to provide a compound or drug product or dosage form containing the compound, other therapy, remedy, or treatment.

As used herein, “dose” means a quantity of drug as per present disclosure, given to the individual for treating or preventing the disease or disorder at one specific time.

As used herein, “in need thereof” is used interchangeably to mean a judgment made by a caregiver (e.g., doctor, physician, nurse, nurse practitioner, pharmacist, etc.) that an individual requires or will benefit from treatment. This judgment is made based on a variety of factors that are in the realm of a caregiver's expertise, but that includes the knowledge that the individual is ill, or will become ill, as the result of a disease, condition, or disorder that is treatable by the compounds of the disclosure.

The term “composition” and “dosage form” as in pharmaceutical composition, is intended to encompass a drug product comprising an endothelin-B receptor agonist or its pharmaceutically acceptable salt thereof and one or more other pharmaceutically acceptable excipients.

The endothelin-B receptor agonist or its pharmaceutically acceptable salt thereof can be administered in the form of a pharmaceutical composition. A variety of administration routes are available. The particular mode selected will depend upon, for example, the particular condition being treated and the dosage required for therapeutic efficacy. The methods of this disclosure may be practiced using any mode of administration that is medically acceptable, meaning any mode that produces effective levels of an immune response without causing clinically unacceptable adverse effects. In some embodiments, the administration is via systemic routes. In some embodiments, the administration is via parenteral routes. The term “parenteral” includes subcutaneous, intravenous, intramuscular, intradermal, and intraperitoneal injection or infusion techniques. In a preferred embodiment, the administration is via intravenous route.

In a preferred embodiment, the composition is a lyophilized powder for injection. As used herein, the term freeze-dried or lyophilized powder or cake or preparation refers to any solid substance that has undergone the process of lyophilization or freeze-drying of aqueous solution. Ideally, a lyophilized preparation is obtained by freeze-drying a solution made up of aqueous solvents.

### Diabetes

Diabetes is a chronic disease that occurs when the body doesn't produce enough insulin or can't use it properly

There are different types of diabetes, including:
Type 1 diabetes
Type 2 diabetes
Maturity-onset diabetes of the young (MODY)
Gestational diabetes
Neonatal diabetes
Steroid-induced diabetes

The present disclosure provides a method of treating or preventing diabetes and its complications in a subject in need thereof comprising administering an endothelin-B receptor agonist or its pharmaceutically acceptable salt thereof.

We started developing sovateltide as a drug to treat cerebral ischemic stroke and conducted an open-label phase I study in India to determine the safety, tolerability, and pharmacodynamics of multiple ascending doses of sovateltide in 9 healthy male volunteers (CTRI/2016/11/007509). The study was designed with three cohorts, each receiving 3 doses of either 0.3, 0.6, or 0.9 μg/kg administered intravenously over a minute. A total of 4 non-serious adverse events (uneasiness, sweating, abdominal discomfort, and vomiting) were reported after the first dose of 0.9 μg/kg of sovateltide. The adverse events were resolved in about 10 minutes without any intervention [29-31]. Subsequently, in a randomized, double-blind trial conducted in India with a limited number (40) of patients, sovateltide showed promising efficacy in patients with acute cerebral ischemic stroke (NCT04046484). Three doses of sovateltide, each dose of 0.3 μg/kg, were administered intravenously at intervals of 3±1 hour on days 1, 3, and 6. The total dose/day was 0.9 μg/kg post-randomization. No sovateltide related adverse event was reported in patients.

An unexpected finding of a decrease in blood glucose was observed in stroke patients treated with sovateltide in the phase II trial conducted in 40 patients. In the control group, patients were treated with saline along with the standard of care for stroke patients, while in the sovateltide group, patients were treated with sovateltide along with the standard of care. In control, the blood glucose decreased from the mean of 178.9 mg/dL to 156.2 mg/dL at 90 days of treatment; therefore, a 22.7 mg/dL decrease was observed. On the other hand, in the sovateltide group, the blood glucose decreased from the mean of 250.7 mg/dL to 141.3 mg/dL at 90 days of treatment; therefore, a 109.3 mg/dL decrease was observed. This was an unexpected finding, and we embarked on further analysis of patients in the phase III trial.

This was a multicenter, randomized, placebo-controlled, double-blind, phase III clinical trial of sovateltide in patients with acute cerebral ischemic stroke receiving the best standard of care (SOC). The study was conducted in 18 centers across India. Patients were randomized in a 1:1 ratio to the sovateltide or the control group. The study drug (normal saline or sovateltide (0.3 μg/kg) was administered intravenously in three doses at intervals of 3±1 hour on days 1, 3, and 6 (total sovateltide dose of 0.9 μg/kg/day). The control group received saline plus SOC, while the sovateltide group received sovateltide treatment plus SOC post-randomization. The study duration for an individual patient was 90 days, including five study visits: visit 1 included screening, randomization, baseline measurements, and treatment on day 1, measurements on day 2, and measurements and treatment on days 3 and 6, after the first visit measurements were included in visit 2 on day 12±2, visit 3 on day 30±5, visit 4 on day 60±7 while visit 5 was at the end of study (day 90±7). This study was conducted in 18 hospitals across India. A total of 158 eligible patients were enrolled in the study. Eighty patients were randomized in the sovateltide group and 78 in the control group (FIG. 1). About 10% (8 out of 78) in the control group and 16% (13 out of 80) in the sovateltide group discontinued the study. In a particular embodiment, diabetes is a major risk factor for cerebral stroke, and patients who had cerebral stroke were treated with sovateltide, an endothelin-B receptor agonist, plus standard of care for cerebral stroke patients showed significantly lower blood glucose than those treated with saline plus standard of care.

At randomization, the blood glucose was 145.5 (95% CI 134.1 to 156.9) mg/dL in the control group and 150.2 (95% CI 132.0 to 168.5) mg/dL in the sovateltide group. However, at 90 days of treatment in the control group, blood glucose was 143.3 (95% CI 126.5 to 160.1) mg/dL, while in the sovateltide group, blood glucose was 125.9 (95% CI 114.5 to 137.2) mg/dL. Hence, saline treatment decreased blood glucose by only 2.2 mg/dL, while sovateltide treatment decreased blood glucose by 24.3 mg/dL.

In diabetic patients, the blood glucose decreased by 10.25 (95% CI −25.08 to 45.58) mg/dL at day 90 from the baseline in saline treated patients, while in the sovateltide treated patients, it decreased by 47.03 (95% CI −90.32to −3.74) mg/dL at day 90 from the baseline, which was statistically significant (p=0.0338). The blood glucose at day 90 was 180.6 (95% CI 151.6 to 209.62) mg/dL in the control group, while it was 157.7 (95% CI 136.4 to 179.0) mg/dL in the sovateltide group.

The unexpected finding of a decrease in blood glucose initially observed in a smaller number of patients was confirmed in a more significant number of patients.

Insulin-secreting pancreatic beta cells are important for regulating glucose metabolism. Loss of beta cells could lead to diabetes. Beta cell regeneration is of great significance in the clinical treatment of diabetes. The major cellular sources of beta cells include endogenous beta cell proliferation, pluripotent stem cell differentiation, and trans-differentiation from other non-beta cells. The existence of endogenous pancreatic stem cells or progenitors in adults remains controversial. In the brain the expression of neuronal differentiation markers Doublecortin (p=0.00011), HuC/HuD (p=0.0037), and NeuroD1 (p=0.00002) was upregulated in the sovateltide compared to the control group. Triphenyl tetrazolium chloride and TUNEL analyses showed reduced infarct volume and DNA damage in the sovateltide group compared to the control group on day 7. Sovateltide treatment also increased mitochondrial fusion (MFN2; p<0.0001), mitochondrial cross-sectional area (p<0.05), and mitochondrial/tissue area ratio (p<0.05). Moreover, increased mitochondrial DNA {MT ATP8} (p=0.0418) was observed, indicating enhanced mitochondrial biogenesis. Sovateltide treated rats performed better in neuro-and motor function tests post-MCAO. After hypoxia, sovateltide treated cultured rat neural progenitor cells (NPCs) also demonstrated higher levels of neuronal differentiation and survival with upregulation of NeuroD1 and NeuN. The study demonstrates a unique mechanism of action of sovateltide involving the differentiation of NPCs into neuronal cells and promoting neural cell survival through upregulating specific neuronal markers. It is possible that in the pancreas, beta cells are regenerated by sovateltide, similar to the effect of sovateltide in neuronal regeneration and repair in the brain. ET-1 (100 nmol/L) stimulated insulin secretion from islets incubated at 8.3, 11.6, 16.7, and 25 mmol/L glucose (P<0.05). BQ123 (ETA antagonist) (10 pmol/L to 10 nmol/L) and BQ3020 (ETB agonist) (1 nmol/L to 1 micromol/L) had no effect on glucose (16.7 mmol/L)-stimulated insulin secretion, but BQ123 counteracted the stimulatory effect of ET-1 (100 nmol/L) at concentrations of 1 nmol/L to 10 micromol/L (P<0.01), indicating involvement of ETA and not ETB receptors (Gregersen and Hermansen (2000) Endothelin-1 (ET-1)-potentiated insulin secretion: involvement of protein kinase C and the ET(A) receptor subtype; Metabolism 49(2): 264-9)

The association between endothelin-1 (ET-1) and obesity has been suggested; however, the types of endogenous receptors, ETA, and ETB involved are unknown. ETA and ETB receptors are present in all insulin-sensitive tissues, including adipose, liver, and muscle (Haley et al., (2020) Endothelin-1 in the pathophysiology of obesity and insulin resistance; Obes Rev. 2020 Dec;21(12):e13086). It is possible that endogenous ET-1 may play a role in the pathogenesis of insulin resistance through ETB receptors, and sovateltide may reduce blood glucose through that mechanism.

A reduction in body weight by sovateltide is possible because food intake may be reduced following treatment with endothelin agonists, including sovateltide. In experimental studies, food intake was reduced in animals treated with sovateltide, which will reduce body weight and may be used to treat obesity.

A patent discloses the methods comprising administering an effective amount of endothelin or an endothelin agonist, alone or in conjunction with other compounds or compositions that affect satiety. There was a dose-dependent reduction in food intake following peripheral administration of ET-1 and ET-2, ET-3, and Sarafotoxin S6c. The effectiveness of ET-3 in reducing food intake suggests that the anoretic effect is likely mediated by ETB receptors since ET-3 binds ETA poorly, if at all. The ETB agonist, adenoregulin infused continuously for 2-4 wk in DIO mice exhibited a sustained weight reducing effect as well as a reduction in body fat (Patent: Endothelin and endothelin receptor agonist for treatment of obesity and related metabolic disorders. World Intellectual Property Organization, WO2007112069 A2 2007-10-04).

The existing data on the interactions among insulin, insulin resistance, and endothelin suggests how those contribute to the development of hypertension in insulin-resistant states. Several mechanisms connect insulin resistance and compensatory hyperinsulinemia with blood pressure elevation in the context of the metabolic syndrome, i.e. sodium retention, sympathetic activation, and impairment of endothelial nitric oxide production. In vitro studies have shown that insulin stimulates both endothelin-1 production and action on the vascular wall. In vivo, high levels of insulin result in an increase in circulating endothelin-1 in healthy individuals, and this effect is also seen in insulin-resistant subjects (Sarafidis and Bakris (2007) Insulin and Endothelin: An Interplay Contributing to Hypertension Development? Get access Arrow; The Journal of Clinical Endocrinology & Metabolism, Volume 92(2), 379-385).

### Pharmaceutical Compositions

In one particular embodiment, endothelin-B receptor agonist or its pharmaceutically acceptable salt thereof is selected from the group consisting of sovateltide, BQ-3020, [Ala1,3,11,15]-endothelin, sarafotoxin S6c, sarafotoxin 6c, or endothelin-3 or any combination thereof.

In another embodiment, endothelin-B receptor agonist or its pharmaceutically acceptable salt thereof is sovateltide or salt thereof.

In one particular embodiment, the endothelin-B receptor agonist or its pharmaceutically acceptable salt thereof is administered in the form of a pharmaceutical composition.

In another embodiment, the endothelin-B receptor agonist or its pharmaceutically acceptable salt thereof is administered in the form of parenteral composition.

In one particular embodiment, the sovateltide or salt thereof is administered in the form of a pharmaceutical composition.

In another embodiment, the sovateltide or salt thereof is administered in the form of parenteral composition.

In another embodiment, the disclosure relates to a pharmaceutical composition comprising an endothelin-B receptor agonist or its pharmaceutically acceptable salt thereof for treating or preventing diabetes and its complications.

In another embodiment, a reduction in body weight by sovateltide is possible because food intake may be reduced following treatment with endothelin agonists, including sovateltide. In experimental studies, food intake was reduced in animals treated with sovateltide, which will reduce body weight and may be used to treat obesity.

In a preferred embodiment, the composition is administered intravenously.

In certain embodiments, the compositions of the disclosure are administered within 1, 2, 3, 4, 5, 6, 7, 8, 9, 10, 11, 12, 13, 14, 15, 16, 17, 18, 19, 20, 21, 22, 23, or 24 hours, or l,2,3,4,5,6,7,8,9, 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12 months, or 1, 2, 3, 4, 5, 6, 7, 8, 9, 10, 11, 12 years of diagnosis.

In one particular embodiment, the composition is a lyophilized powder for injection.

In another embodiment, the pharmaceutical composition comprises one or more other pharmaceutically excipients.

In one particular embodiment, the composition comprises:
a) endothelin-B receptor agonist or its pharmaceutically acceptable salt thereof present in an amount ranging from about 0.01% to about 10% w/w, and
b) one or more pharmaceutically acceptable excipients.

In another embodiment, the composition comprises:
a) endothelin-B receptor agonist or its pharmaceutically acceptable salt thereof present in an amount ranging from about 0.01% to about 0.02% w/w, and
b) one or more pharmaceutically acceptable excipients.

In one particular embodiment, the composition comprises:
a) sovateltide or salt thereof present in an amount ranging from about 0.01% to about 10% w/w, and
b) one or more pharmaceutically acceptable excipients.

In another embodiment, the composition comprises:
a) sovateltide or salt thereof present in an amount ranging from about 0.01% to about 0.02% w/w, and
b) one or more pharmaceutically acceptable excipients.

In another embodiment, one or more pharmaceutically acceptable excipients are present in an amount ranging from about 10% to about 99% w/w.

In another embodiment, one or more pharmaceutically acceptable excipients are present in an amount ranging from about 10% to about 95% w/w.

In another embodiment, one or more pharmaceutically acceptable excipients are selected from the group consisting of, pH adjusting agents, buffering agents, diluents, tonicity adjusting agents, suspending agents, complexing agents, preservatives, antioxidants, and a combination thereof.

In another embodiment, the pH adjusting agent is selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof.

In one particular embodiment, the pH adjusting agent is sodium citrate. In another particular embodiment, the pH adjusting agent is trisodium citrate. In another particular embodiment, the pH adjusting agent is trisodium citrate dihydrate.

The buffering agent may be any acceptable buffer known in the pharmaceutical arts for the indicated pH range and physiological compatibility. In one embodiment, the buffer comprises an ascorbate, sorbate, formate, lactate, fumarate, tartrate, glutamate, acetate, benzoate, citrate,, gluconate, histidine, glycine, lysine, malate, phosphate or succinate buffer. In another embodiment, the buffer comprises an ascorbate, lactate, tartrate, citrate, gluconate, malate, isocitrate or 2-hydroxybutyrate buffer.

In certain embodiment, the buffering agent can act as pH adjusting agent. In certain embodiment, the pH adjusting agent can act as a buffering agent.

In another embodiment, the tonicity adjusting agent is selected from the group consisting of, sodium acetate, sodium chloride, dextrose, sodium lactate, calcium chloride, sodium bicarbonate, potassium chloride, and a combination thereof.

In another embodiment, the diluent is selected from the group consisting of, mannitol, sorbitol, sucrose, lactose, cyclodextrin, and a combination thereof.

In another embodiment, the diluent is selected from the group consisting of, sodium chloride, Trisodium citrate, Citric Acid anhydrous, Dibasic sodium citrate, Dibasic sodium Phosphate, Hydroxypropyl-cyclodextrin, and mannitol and a combination thereof.

In one particular embodiment, the diluent is mannitol.

In one embodiment, the composition comprises:
a) endothelin-B receptor agonist or its pharmaceutically acceptable salt thereof present in an amount ranging from about 0.01% to about 0.02% w/w, and
b) about 0.05% to about 50% w/w of one or more pH adjusting agents selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof, and
c) about 10% to about 90% w/w of one or more diluents selected from the group consisting of mannitol, sorbitol, sucrose, lactose, cyclodextrin, and a combination thereof.

In another embodiment, the composition comprises:
a) endothelin-B receptor agonist or its pharmaceutically acceptable salt thereof present in an amount ranging from about 0.01% to about 0.02% w/w, and
b) about 0.05% to about 30% w/w of one or more pH adjusting agents selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof, and
c) about 30% to about 80% w/w of one or more diluents selected from the group consisting of mannitol, sorbitol, sucrose, lactose, cyclodextrin, and a combination thereof.

In another embodiment, the composition comprises:
a) endothelin-B receptor agonist or its pharmaceutically acceptable salt thereof present in an amount ranging from about 0.01% to about 0.02% w/w, and
b) about 0.05% to about 30% w/w of sodium citrate, and
c) about 30% to about 80% w/w of mannitol.

In another embodiment, the composition comprises:
a) sovateltide or salt thereof present in an amount ranging from about 0.01% to about 0.02% w/w, and
b) about 0.05% to about 50% w/w of one or more pH adjusting agents selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof, and
c) about 10% to about 90% w/w of one or more diluents selected from the group consisting of mannitol, sorbitol, sucrose, lactose, cyclodextrin, and a combination thereof.

In another embodiment, the composition comprises:
a) sovateltide or salt thereof present in an amount ranging from about 0.01% to about 0.02% w/w, and
b) about 0.05% to about 30% w/w of one or more pH adjusting agents selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof, and
c) about 30% to about 80% w/w of one or more diluents selected from the group consisting of mannitol, sorbitol, sucrose, lactose, cyclodextrin, and a combination thereof.

In another embodiment, the composition comprises:
a) sovateltide or salt thereof present in an amount ranging from about 0.01% to about 0.02% w/w, and
b) about 0.05% to about 30% w/w of sodium citrate, and
c) about 30% to about 80% w/w of mannitol.

In another embodiment, the composition is reconstituted with water for injection in the range of 1-2% w/w.

In one particular embodiment, the lyophilized composition is reconstituted before administration.

In another embodiment, the lyophilized composition is reconstituted before administration with one or more solvents.

In another embodiment, the reconstitution comprises using a sterile physiological solution.

In another embodiment, the composition is reconstituted with water for injection or 0.9% sodium chloride aqueous solution.

In another embodiment, the pH of the reconstituted composition is from about 5.0 to about 8.5.

In one particular embodiment, the pH of the reconstituted composition is from about 5.0 to about 7.5.

In another particular embodiment, the pH of the reconstituted composition is from about 7.0 to about 8.5.

In another embodiment, the osmolality is between 240 to 310 mOsm/L for the reconstituted liquid composition and preferably about 286 mOsm/L.

In one embodiment, the present disclosure provides a kit or a co-pack comprising a vial containing lyophilized pharmaceutical composition and an ampoule/vials containing 0.9% sodium chloride aqueous solution or water for injection.

### Process Embodiments

In one particular embodiment, the present disclosure provides a method of preparing pharmaceutical composition, wherein the composition is prepared by a process comprising the steps of: dissolving endothelin-B receptor agonist or its pharmaceutically acceptable salt thereof, and one or more other pharmaceutically excipients such as diluent, pH adjusting agent in a solvent; filtering to eliminate any bacteria or other contaminants; filling into sterile containers up to the target fill volume; and lyophilizing.

In another embodiment, the present disclosure provides a method of preparing pharmaceutical composition, wherein the composition is prepared by a process comprising the steps of: dissolving diluent in water for injection, adding endothelin-B receptor agonist or its pharmaceutically acceptable salt thereof and pH adjusting agent, checking and adjusting the pH using pH adjusting agent from about 5.0 to 8.5, filtering to eliminate any bacteria or other contaminants; filling into sterile containers up to the target fill volume; and lyophilizing.

In another embodiment, the present disclosure provides a method of preparing pharmaceutical composition, wherein the composition is prepared by a process comprising: dissolving pH adjusting agent in the water for injection in order to obtain a mixture; adding the endothelin-B receptor agonist or its pharmaceutically acceptable salt thereof in the mixture followed by addition of diluent in order to obtain a solution; checking the pH of the solution, for making the volume of the solution, followed by sterile filtering the solution; filling sterile filtered solution in vials; half stoppering the plugs on the vials; loading the vials in freeze dryer for lyophilization to obtain the lyophilized-based injectable formulation.

In another embodiment, the present disclosure provides a method of preparing a pharmaceutical composition, wherein the composition is prepared by a process comprising: dissolving endothelin-B receptor agonist or its pharmaceutically acceptable salt thereof, pH adjusting agent and diluent in water for injection; filtering the solution through 0.2μ membrane filter; filling the individual vials up to the target fill volume; and lyophilizing of the filled vials.

In another embodiment, the solution in step i) is stirred at 300-350 rpm. In yet another embodiment, the pH of the solution in step i) is adjusted to a pH of about 5.0 to 8.5, preferably about 7.0 to 8.5. In another embodiment, the present disclosure provides a method of preparing a pharmaceutical composition, wherein the composition is prepared by a process comprising: dissolving pH adjusting agent in the water for injection in order to obtain a mixture; adding the endothelin-B receptor agonist or its pharmaceutically acceptable salt thereof in the mixture followed by addition of the diluent in order to obtain a solution; checking the pH of the solution, for making the volume of the solution, followed by sterile filtering the solution and sterile filling the filtered solution in vials; half stoppering of plugs on the vials followed by loading the vials in freeze dryer for lyophilizing to obtain the formulation.

### Dose & Administration

In one particular embodiment, the endothelin-B receptor agonist or its pharmaceutically acceptable salt thereof is administered alone.

In another particular embodiment, the endothelin-B receptor agonist or its pharmaceutically acceptable salt thereof is administered as an adjunctive therapy to existing medicaments.

In another particular embodiment, the endothelin-B receptor agonist or its pharmaceutically acceptable salt thereof is administered as an adjunctive therapy to existing anti-diabetic medicaments.

In one particular embodiment, the sovateltide or salt thereof is administered alone.

In another particular embodiment, the sovateltide or salt thereof is administered as an adjunctive therapy to existing medicaments.

In some embodiments, the subject is receiving or has received an anti-diabetic medicament.

In another particular embodiment, the sovateltide or salt thereof is administered as an adjunctive therapy to existing anti-diabetic medicaments.

In some embodiments, the subject has previously received an anti-diabetic treatment such as an anti-diabetic medicament.

In some embodiments, the anti-diabetic medicament is selected from the commonly used classes of non-insulin medications, including Metformin; Dipeptidyl peptidase 4 (DPP-4) inhibitors; Glucagon-like peptide 1 (GLP-1) and dual GLP-1/gastric inhibitory peptide (GIP) receptor agonists; Sodium-glucose cotransporter 2 (SGLT2) inhibitors; Sulfonylureas; Thiazolidinediones (TZDs). Insulin is also used to treat diabetes.

Sulfonylureas: These stimulate the pancreas to release more insulin. Examples include glimepiride (Amaryl), glipizide (Glucotrol), and glyburide (Micronase)

Thiazolidinediones (TZDs): These help insulin work better in muscles and fat, and reduce glucose production in the liver. Examples include rosiglitazone (Avandia) and pioglitazone (Actos).

SGLT2 inhibitors: These block the body's ability to reabsorb glucose, causing it to be excreted in urine. Examples include canagliflozin (Invokana), dapagliflozin (Farxiga), and empagliflozin (Jardiance).

GLP-1 receptor agonists: These include dulaglutide (Trulicity), exenatide (Byetta), liraglutide (Saxenda), lixisenatide (Adlyxin), and semaglutide (Ozempic).

Anti-diabetic medications can help treat diabetes by lowering blood sugar levels and relieving symptoms. They can also help prevent long-term complications like kidney disease, nerve damage, and retinal damage.

Diabetes is a progressive disease, and medications sometimes stop working as well over time. When this happens, adjustments to your medication or combination therapy can help, including adding insulin to the treatment plan. In some embodiments, the diabetes is resistant to any anti-diabetic medications, and adding sovateltide may help restore sensitivity.

In one embodiment, a reduction in body weight by sovateltide is possible because food intake may be reduced following treatment with endothelin agonists, including sovateltide. In experimental studies, food intake was reduced in animals treated with sovateltide, which will reduce body weight and may be used to treat obesity.

The present disclosure may involve one or more embodiments. It is to be understood that the embodiments below are illustrative of the present disclosure and are not intended to limit the claims to the specific embodiments exemplified. It is also to be understood that the embodiments defined herein may be used independently or in conjunction with any definition, any other embodiment defined herein. Thus, the disclosure contemplates all possible combinations and permutations of the various independently described embodiments. The following examples serve to illustrate the disclosure without limiting the disclosure in its scope.

Moreover, unless stated otherwise, any feature disclosed herein may be replaced by an alternative feature serving the same or a similar purpose.

1. A pharmaceutical composition for treating or preventing diabetes or diabetes-related disorders, comprising;
(a) ETB receptor agonist or its analogues in a predefined amount;
(b) an anti-diabetic medicament is selected from the commonly used classes of non-insulin medications, including Metformin; Dipeptidyl peptidase 4 (DPP-4) inhibitors; Glucagon-like peptide 1 (GLP-1) and dual GLP-1/gastric inhibitory peptide (GIP) receptor agonists; Sodium-glucose cotransporter 2 (SGLT2) inhibitors; Sulfonylureas; Thiazolidinediones (TZDs) or Insulin and its formulations or a mixture thereof; and
(c) one or more excipients.

2. A method of preventing or treating diabetes, a disease or diabetes-related disorder, by administering a therapeutically effective amount of an endothelin B (ETB) receptor agonist to an individual in need thereof.

3. The method of claim 2, wherein the ETB receptor agonist is selected from the group consisting of N-Succinyl-[Glu9, Ala11,15] endothelin 1 (IRL-1620), BQ-3020, [Ala1,3,11,15]-endothelin, sarafotoxin S6c, and endothelin 3.

4. The method of claim 2, wherein the ETB receptor agonist is administered at a dose ranging from about 0.0001 mg/kg to about 0.5 mg/kg.

5. The method of claim 2, wherein the administering comprises multiple doses of the ETB receptor agonist.

6. The method of claim 2, wherein the administering comprises a single dose of the ETB receptor agonist.

7. The method of claim 2, wherein ETB receptor agonist or its analogues are delivered intravenously, orally, intramuscularly, subcutaneously by procedures through direct injections, osmotic mini-pumps and reciprocating perfusion systems.

8. The method of claim 2, wherein ETB receptor agonist or its analogues are conjugated with either microparticles or nanoparticles.

9. The method of claim 2, wherein the dosage of ETB receptor agonist or its analogues may be administered once or multiple times in a day or in weeks or in months.

10. A pharmaceutical formulation, comprising:
a) endothelin-B receptor agonist in an amount ranging from about 0.01% to about 0.02% w/w;
b) about 0.05% to about 50% w/w of one or more pH adjusting agents; and
c) about 10% to about 90% w/w of one or more diluents.

11. The formulation of claim 10, wherein the endothelin-B receptor agonist is sovateltide or a salt thereof.

12. The formulation of claim 10, wherein the pH adjusting agents selected from the group consisting of, sodium phosphate, citric acid, sodium citrate, sodium bicarbonate, glacial acetic acid, hydrochloric acid, sodium hydroxide, meglumine, histidine monohydrochloride, tris(hydroxymethyl)aminomethane, maleate and a combination thereof.

13. The formulation of claim 10, wherein the pH adjusting agent is sodium citrate.

14. The formulation of claim 10, wherein the diluents selected from the group consisting of mannitol, sorbitol, sucrose, lactose, cyclodextrin, and a combination thereof.

15. The formulation of claim 10, wherein the diluents is mannitol.

16. The formulation of claim 10, wherein the composition further comprises sodium chloride.

17. The formulation of claim 10, wherein the composition further comprises water.

18. The formulation of claim 10, wherein the formulation is lyophilized and comprises:
a. sovateltide or salt thereof present in an amount ranging from about 0.01% to about 0.02% w/w;
b. about 0.05% to about 30% w/w of sodium citrate; and
c. about 30% to about 80% w/w of mannitol.

19. The lyophilized formulation of claim 18, wherein it is reconstituted by water or 0.9% sodium chloride solution.

20. A method of treating diabetes, a disease or disorder caused by diabetes, comprising administering the lyophilized pharmaceutical formulation of claim 18, along with an anti-diabetic agent to a subject in need thereof.
